(19) United States
(12) Patent Application Publication
(10) Pub. No.: US 2003/0040642 A1
(43) Pub. Date: Feb. 27, 2003

(54) Method for adjusting concentration of starting materials in gas phase contact reaction process, method for controlling reaction process by the adjusting method, and process for producing lower fatty acid ester using the control method

(76) Inventors: Hidetoshi Goto, Oita-shi (JP); Shigeru Hatanaka, Oita-shi (JP)

Correspondence Address: Sughrue Mion Zinn, Macpeak & Seas, Suite 800, 2100 Pennsylvania Avenue NW, Washington, DC, 20037-3213, US

(21) Appl. No.: 10/018,140

(22) PCT Filed: Feb. 23, 2001
(86) PCT No.: PCT/JP01/01388

(30) Foreign Application Priority Data
Feb. 24, 2000 (JP) 2000-046667

(51) Int. Cl. C07C 51/16

(52) U.S. Cl. 562/544

(57) ABSTRACT

A method for adjusting the concentration of starting materials, comprising adjusting the starting material concentration in a gas fed to a reactor in a gas phase contact reaction process having a recycling system, wherein the concentration of a starting material in a gas in the process is measured, the starting material is fed by setting the feed amount of the starting material newly added to the process based on the measured value, and thereby the starting material concentration in the gas fed to the reactor is controlled; and a method for controlling a reaction process using the above-described adjusting method.

### CROSS-REFERENCE TO RELATED APPLICATIONS

This application is an application filed under 35 U.S.C. §111(a) claiming benefit, pursuant to 35 U.S.C. §119(e)(1), of the filing date of the Provisional Application 60/256,914 filed Dec. 21, 2000, pursuant to 35 U.S.C. §111(b).

### TECHNICAL FIELD

The present invention relates to a method for adjusting the concentration of starting materials in a gas phase contact reaction process, a method for controlling a reaction process by the adjusting method, and a process for producing a lower fatty acid or a lower fatty acid ester using the control method.

More specifically, the present invention relates to a method for adjusting the concentration of starting materials, wherein in a gas phase contact reaction process having a so-called recycling system of additionally feeding a fresh starting material to a gas containing unreacted starting material after passing through a reactor where a gas phase contact reaction is performed, and again returning the gas to the reactor, the feed amount of the fresh starting material is determined based on the concentration of the starting material in the gas measured at an arbitrary site in the process and thereby the concentration of the starting material in the gas fed to the reactor is adjusted; a method for stably controlling the reaction process itself by the adjusting method; and a process for producing a lower fatty acid or a lower fatty acid ester using the control method.

### BACKGROUND ART

In a process using a reactor for performing a reaction in the form of a gas phase contact reaction, the gas fed to the reactor generally passes only once through the reactor and there is not known a case where the starting material in the gas is thoroughly reacted to obtain an objective substance. In particular, when two or more starting materials are used, one or more starting material is used in excess of the theoretical amount to improve the reaction results such as conversion of starting material and selectivity to the objective substance in the reaction or for the purpose of stably controlling the reaction. In such a case, recovery and re-use of the starting material used in excess is usually an essential matter in view of profitability in the practice of the process.

Accordingly, in a process using a reactor for performing a reaction in the form of a gas phase contact reaction, the reaction process generally has a so-called recycling system where, after the passing of a gas containing a starting material through the reactor, the objective substance is separated (and if desired, refined), then a fresh starting material is additionally fed to the gas containing unreacted starting material to adjust the concentration of the starting material in the gas, and the gas is again returned to the reactor and reacted.

In order to control such a reaction process, namely, in order to maintain the reaction results or stabilize the reaction, so-called “process operating conditions” such as various reaction conditions must be controlled. In particular, it is important to control the concentration and compositional ratio of the starting material in the gas fed to the reactor and the pressure within the system, particularly within the reactor.

Generally, in the process of a gas phase contact reaction, the presence of so-called “inert material” having no relation to the reaction cannot be neglected.

The inert material in general is considered to include:

1) a non-objective substance generated as a by-product in the reaction,

2) a substance contained as an impurity in the starting material itself,

3) an inert gas added to adjust the concentration of the starting material for the purpose of controlling the reaction,

and the like.

These non-objective substance, impurity and inert gas accumulate in the system in the course of repetition of the recycling operation in the reaction process having the above-described recycling system, and this causes a relative reduction in the concentration of the starting material from the standpoint of reaction, or elevation of the pressure within the system.

Therefore, in the reaction process having a recycling system, a so-called “purging operation” of expelling a part of the gas within the system to the outside of the system is performed and one purpose of this operation is to discharge the inert material out of the system and thereby prevent the pressure within the system from elevating.

Another purpose of the purging operation in the reaction process having a recycling system is to control the feed amount of the starting material at the time of newly adding this due to consumption in the reaction, thereby to adjust the concentration of the starting material in the gas fed to the reactor, and in turn to control the reaction process itself.

A very large number of reports are known on such a process of preventing the elevation of pressure within the system by the purging operation and at the same time adjusting the concentration of starting materials. Specific examples thereof include Tasuku Senbon and Futoshi Hanabuchi, Keiso System no Kiso to Oyo, “Dai 9-Sho, Process Unit no Seigyo (Oyo 1), Dai 6-Ko, Recycle Hanno-Kei no Seigyo” (Introduction and Application of Instrumentation System, “Chap. 9—Control of Process Unit (Application I), Item 6(e)—Control of Recycling Reaction System”), 1st ed., 9th imp., pp. 530-531, Ohmu Sha (Oct. 20, 1993).

According to the general discussion of this publication, it is stated that the amount of gas expelled by the purging operation (hereinafter simply referred to as “purge amount”) is determined based on the value of a concentration controller so that the concentration of the inert material within the system can be maintained constant or, by supposing that the elevation of pressure within the system is the increase of inert material, the purge amount is determined by the value of a pressure controller used in place of the concentration controller.

In the former case, when the concentration controller detects the fact that the concentration of inert material in the gas is elevated, the operation is directed to increase the purge amount, as a result, the discharge of inert material is accelerated and the concentration of inert material within the system gradually decreases. The operation directed to increase the purge amount is, at the same time, an operation directed to decrease the pressure within the system and therefore, a fresh starting material is additionally fed to the system, as a result, the concentration of the starting material in the gas increases.

In the latter case, when the pressure controller detects the increase of pressure, the purge amount is increased to reduce the pressure and increase the discharge of inert material. At the same time, in order to compensate for the reduction of pressure accompanying this, a fresh starting material is additionally fed to the system and similarly to the former case, the concentration of the starting material in the gas increases.

In either case, by controlling the purge amount, the inert material concentration within the system is controlled, in other words, the concentration of starting materials in a gas fed to the reactor is adjusted and at the same time, the pressure within the system is adjusted, whereby the reaction is in turn controlled.

In such a method, when an equilibrium is reached between the amount of inert material produced or carried over in the system and the amount of inert material discharged out of the system by the purging operation, the concentration of the starting material in the gas and the pressure within the system each equilibrate at a certain point and, as a result, the reaction and process as the whole is put into a stationary state and stabilized.

Furthermore, this method is advantageous in that the feed amount of the fresh starting material additionally fed is automatically adjusted by the pressure within the system and therefore, means for adjusting the feed amount of the starting material, such as control valve, can be omitted. An adjusting means such as control valve is also not used in the process example described in the above-described general discussion.

As such, in the process using a reactor for performing a reaction in the form of a gas phase contact reaction and having a recycling system, it is very common to utilize a purging operation for the adjustment of the concentration of starting materials in a gas fed to the reactor and, by virtue of the adjusted concentration of starting materials, control the reaction to proceed in a stable state and yield good results.

However, depending on the properties of the catalyst used in the reaction, satisfactory control may not be attained, in some cases, by this method. Specific examples of such a case include a process using a catalyst which requires the concentration of the starting material to fall in a very narrow range for achieving a stable reaction and good reaction results.

In a process using a catalyst having such properties, the concentration of the starting material in a gas fed to the reactor must be adjusted with precision so as to keep the concentration in the required narrow range. However, the above-described conventional method, namely, a method of adjusting the concentration of starting materials by the purge amount, and thereby controlling a reaction, has the following problems.

When a change appears in the concentration of the inert material or the starting material, the control of the reaction is already deviated from the optimum value and the deviation is made larger in view of the properties of the catalyst. Therefore, the optimum value must be rapidly regained and, for this purpose, the concentration of the starting material must be changed so as to satisfactorily cancel the change detected, but setting the purge amount to realize this is difficult. Moreover, the relationship between the change in the concentration of the starting material and the purge amount is not constant but fluctuates accompanying the deactivation of catalyst or a change in the load on process, therefore, the adjustment must be done on many occasions and this is not practical.

Furthermore, in the method of operating the purge amount and thereby controlling the amount of the starting material additionally fed for compensating for the reduction of pressure accompanying it, a time lag is generally liable to occur in proceeding through operation of the purge flow rate-change in the pressure within the system-change in the amount of starting material additionally fed-change in the concentration of starting material and therefore, the control cannot be performed with good precision.

In addition, the purge amount itself is usually smaller than the held amount within the system and the variation of the purge amount caused so as to adjust the concentration of the starting material is even smaller, therefore, the effect of the purge operation on the pressure is very slight. As a result, the pressure within the system changes by a small amount, and slowly, due to the purge operation and it is very difficult to detect this slight change in the pressure with good precision, to set the amount of the fresh starting material additionally fed so as to recover the preferred concentration of the starting material, and to have this reflected in the process.

In the above, one example has been described showing that in the reaction process using a reactor of performing a reaction in the form of a gas phase contact reaction and having a recycling system, optimal control cannot be hardly attained by the method heretofore commonly used where the control of the concentration of starting materials in the reactor and the adjustment of the pressure within the system are performed by the control of the purge amount. Of course, the application example of the present invention is not limited to the above-described example with respect to the properties of the catalyst.

### DISCLOSURE OF INVENTION

The present inventors have made extensive investigations on the method for adjusting the concentration of starting materials and the method for controlling the reaction process, in a process using a catalyst difficult to control by a commonly used method such that, in the reaction process using a reactor of performing a reaction in the form of a gas phase contact reaction and having a recycling system, the purge amount is controlled to control the amount of the starting material additionally fed to the reactor, to thereby adjust the concentration of the starting material, to simultaneously adjust the pressure within the system and, in turn, to control the reaction process. As a result, the present invention has been accomplished.

More specifically, the present invention (I) is a method for adjusting the concentration of starting materials in a gas fed to a reactor in a gas phase contact reaction process having a recycling system, the method comprising measuring the concentration of a starting material in a gas in the process, feeding the starting material by setting the feed amount of the starting material newly added to the process based on the measured value, and thereby controlling the concentration of the starting material in a gas fed to the reactor.

The present invention (II) is a method for controlling a reaction process, comprising controlling a gas phase contact reaction process, wherein at least one control method is the method for adjusting the concentration of starting materials of the present invention (I).

The present invention (III) is a process for producing a lower fatty acid from a lower olefin and oxygen in the presence of a catalyst, which is controlled by the method for controlling a process of the present invention (II).

The present invention (IV) is a process for producing a lower fatty acid ester from a lower olefin and a lower fatty acid in the presence of a catalyst, which is controlled by the method for controlling a process of the present invention (II).

The present invention (V) is a process for producing a lower fatty acid ester from a lower olefin, oxygen and a lower fatty acid in the presence of a catalyst, which is controlled by the method for controlling a process of the present invention (II).

### BRIEF DESCRIPTION OF DRAWINGS

The figures are each a process diagram showing one embodiment in the practice of the present invention, a schematic view of an experimental apparatus used in Examples and Comparative Examples, or a graph showing the results in Examples and Comparative Examples.

FIG. 1 is a conceptual view for explaining the concept of the present invention. The numerical references in the Figure indicate the following parts.

1: reaction device

2: device for separating product from others

3: product line

4: purge line

5: starting material feed line

FIG. 2 is a schematic view of the apparatus used in Examples 1 and 2. The solid line, the dotted line and the single dashed line indicate a process line, a signal line and a control line, respectively. The numerical references in the Figure indicate the following parts.

6: reactor

7: gas-liquid separation device

8: level control device

9: acetic acid fetch line

10: pressure control device

11: purge line

12: ethylene concentration analyzer

13: flow meter

14: oxygen flow rate control device

15: oxygen feed line

16: ethylene flow rate control device

17: ethylene feed line

18: concentration controller

19: computing device

FIG. 3 is a schematic view of the apparatus used in Comparative Example 1. The solid line, the dotted line and the single dashed line indicate a process line, a signal line and a control line, respectively. The numerical references in the Figure are the same as those in FIG. 2 except for the following.

20: pressure gauge

21: flow meter

FIG. 4 is a schematic view of the apparatus used in Comparative Example 2. The solid line, the dotted line and the single dashed line indicate a process line, a signal line and a control line, respectively. The numerical references in the Figure are the same as those in FIG. 2 except for the following.

21: flow meter

22: densitometer

FIG. 5 is a schematic view of the apparatus used in Example 3. The solid line, the dotted line and the single dashed line indicate a process line, a signal line and a control line, respectively. The numerical references in the Figure are the same as those in FIG. 2 except for the following.

23: acetic acid flow rate control device

24: acetic acid feed line

25: computing device

FIG. 6 is a schematic view of the apparatus used in Example 4. The solid line, the dotted line and the single dashed line indicate a process line, a signal line and a control line, respectively. The numerical references in the Figure are the same as those in FIG. 2 except for the following.

26: propylene flow rate control device

27: propylene feed line

28: acetic acid flow rate control device

29: acetic acid feed line

30: propylene concentration measuring device

31: computing device

FIG. 7 is a schematic view of the apparatus used in Example 5. The solid line, the dotted line and the single dashed line indicate a process line, a signal line and a control line, respectively. The numerical references in the Figure are the same as those in FIG. 2 except for the following.

28: acetic acid flow rate control device

29: acetic acid feed line

32: computing device

FIG. 8 is a graph showing the results in Example 1.

FIG. 9 is a graph showing the pressure results in Example 1.

FIG. 10 is a graph showing the results in Example 2.

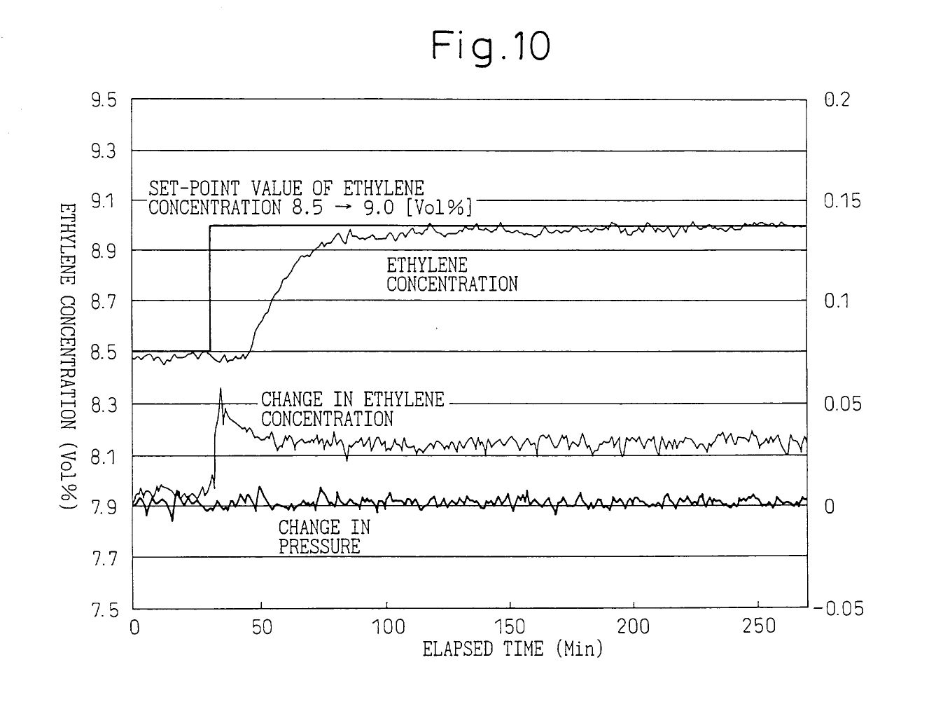

FIG. 11 is a graph showing the results in Comparative Example 1.

FIG. 12 is a graph showing the results in Comparative Example 2.

FIG. 13 is a graph showing the pressure results in Comparative Example 2.

FIG. 14 is a graph showing the results in Example 3.

FIG. 15 is a graph showing the results in Example 4.

FIG. 16 is a graph showing the results in Example 4.

FIG. 17 is a graph showing the results in Example 5.

### BEST MODE FOR CARRYING OUT THE INVENTION

The present invention is described in detail below.

The present invention (I) is a method for adjusting the concentration of starting materials in a gas fed to a reactor in a gas phase contact reaction process having a recycling system, the method comprising measuring the concentration of a starting material in a gas in the process, feeding the starting material by setting the feed amount of the starting material newly added to the process based on the measured value, and thereby controlling the concentration of the starting material in a gas fed to the reactor.

According to the method for adjusting the concentration of starting materials in a gas fed to a reactor in a gas phase contact reaction process of the present invention (I), the concentration of starting materials in a gas fed to a reactor in a gas phase contact reaction process having a recycling system is not adjusted by a conventionally used indirect method, namely, by replenishing a starting material for compensating the reduction in pressure within the system due to the purging operation and thereby maintaining the pressure, but is adjusted by a direct method such that the starting material concentration is measured at an arbitrary position in the process and the starting material is fed after the feed amount of the starting material newly added is determined based on the measured value.

The present invention (I) can be applied to any reaction process without any particular limitation as long as it is a gas phase contact reaction process having a recycling system. Needless to say, in the case where the concentration of starting materials cannot be appropriately adjusted by the conventional purging operation because of various reasons, and even in the case where the purging operation is practically satisfied, the present invention can be applied so as to attain more efficient control of the reaction. Specific examples of the process to which the present invention can be particularly preferably applied include a process for producing a lower fatty acid from a lower olefin and oxygen in the presence of a catalyst and a process for producing a lower fatty acid ester from a lower olefin and a lower fatty acid in the presence of a catalyst.

The rector for use in the gas phase contact reaction of the present invention (I) is not particularly limited. In a preferred embodiment, the reaction is a fixed bed gas phase contact reaction and in a more preferred embodiment, the reactor has a multitubular form and/or a multilayer form. In general, a reactor having a multitubular form and/or a multilayer form is superior in the reaction results, thermal efficiency and ease of control. Of course, the reactor form is not limited thereto.

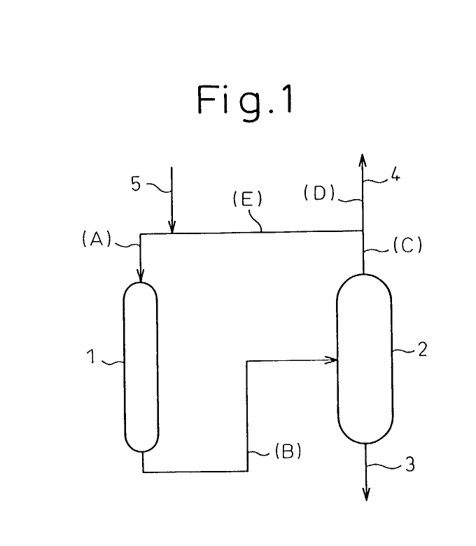

In the present invention (I), the position of measuring the concentration of a starting material in the gas is not particularly limited and may be performed at any site of the process. FIG. 1 is a conceptual view showing one example of the process. In this example, the measurement of concentration may be performed at any site on the following characteristic lines:

(A) a line immediately before the reactor, where a fresh starting material is added to the recycle gas,

(B) a line from the reactor outlet to the separation device,

(C) an outlet line from the separation device,

(D) a purge line,

(E) a recycle gas line before a fresh starting material is added, and the like.

Among these, (A) and (D) are preferred as the position for measuring the concentration of a starting material. (A) is preferred because the starting material measured contains the starting material newly added immediately before the reactor and the concentration of the starting material is least prone to disturbance. (D) is preferred because the gas after the measurement can be wholly introduced into the purge line and therefore, a measuring method having a possibility of affecting the starting material concentration itself can be used. However, whichever position of (A) to (E) is used for the measurement, the amount of the starting material newly added can be calculated from the measured value and therefore, the measuring site is not limited to these positions.

The measuring method of the starting material concentration is not particularly limited and a method commonly used for measuring the gas component concentration may be used. Specific examples thereof include a method of providing a sampling valve at the site of measuring the concentration, introducing a part of the gas into a gas chromatograph and measuring the compositional ratio by appropriately combining a column and a detector, and a method of providing a cell in the line itself and spectro-optically measuring the concentration using a specific wavelength, however, the present invention is not limited thereto. The measuring conditions are preferably such that an exact concentration can be measured within a short time in view of the properties of the starting material to be measured and that other components as inclusions are prevented from exerting their effect.

The method for adjusting the amount of the starting material additionally fed to the process is not particularly limited and a method heretofore commonly used for adjusting the feed amount of a starting material can be used. To speak specifically, adjusting means provided in the starting material feed line, such as a flow rate adjusting valve, can be used.

The amount of the starting material additionally fed according to the measured concentration of the starting material can be controlled by a commonly used control method. Specifically, a method of converting the concentration value of the starting material measured by the above-described method into a certain kind of an electric signal and in accordance with the change in the signal, controlling the adjusting means provided in the starting material feed line to determine the amount of the starting material additionally fed, may be used.

More specifically, a method where the target concentration of a starting material is compared with the concentration of the starting material measured at the reactor inlet and then the starting material feed amount is automatically operated so as to cancel the deviation, may be used. Examples of the automatic operation apparatus which can be used include a feed back control apparatus having respective operations of proportional, integral and derivative (hereinafter simply referred to as a “PID controller”). In the case of using the PID controller as the concentration controller, a technique of regarding the concentration of a starting material in a gas as the process variable (hereinafter simply referred to as “PV”) and directly operating the starting material feed amount adjusting valve based on the manipulated variable (hereinafter simply referred to as “MV”) may be used.

For locally limiting the disturbance which may occur in the feed amount of the starting material, means called a concentration-flow rate cascade control construction may be used, where the PID controller is provided also to the starting material feed amount adjusting valve and the MV of the concentration controller is set as a set-point value (hereinafter simply referred to as “SV”) of the starting material feed amount controller, and by using this construction, more excellent results can be obtained.

Of course, a method of separately computing a desired concentration of the starting material based on the material balance of substances and adjusting the starting material feed amount according to the computed value may be used and, furthermore, a so-called advanced control system represented by model predictive control and fuzzy control may also be used. In general, the PID controller is used because this is simple and convenient.

The present invention (II) is described below. The present invention (II) is a method for controlling a reaction process, comprising controlling a gas phase contact reaction process, wherein at least one control method is the method for adjusting the concentration of starting materials of the present invention (I).

According to the method for controlling a reaction process of the present invention (II), specific methods for stably controlling a general gas-phase contact reaction process contain the method for adjusting the concentration of starting materials of the present invention (I). In other words, the present invention (II) is a method for stably and efficiently controlling the entire process by means of the method for adjusting the concentration of starting materials shown in the present invention (I).

Generally, in order to control a reaction process, namely, in order to maintain the reaction results or stabilize the reaction, so-called “process operating conditions” such as various reaction conditions must be controlled. In particular, it is important for maintaining the reaction results to control the concentration of the starting material in the gas fed to a reactor. In the method for controlling a process of the present invention (II), the method for adjusting the concentration of starting materials in a gas fed to a reactor in a gas phase contact reaction process of the present invention (I) is used and this adjusting method is characterized in that the starting material concentration necessary for the stable control of the process is not adjusted by a conventionally used indirect method, namely, by replenishing a starting material for compensating the reduction in pressure within the system due to the purging operation and thereby maintaining the pressure, but is adjusted by a direct method such that the starting material concentration is measured at an arbitrary position of the process and the starting material is fed after the feed amount of the starting material newly added is determined based on the measured value.

The reaction process to which the present invention (II) can be applied, the site and method for measuring the starting material concentration, and the method for controlling the feed amount of the newly added starting material based on the measured value are the same as those in the present invention (I).

In the process control method of the present invention (II), other process operating conditions may be contained as long as these do not impair the effect of the method for adjusting the concentration of starting materials of the present invention (I). Specific examples thereof include the reactor temperature, the flow rate of a gas containing the starting material, and the separation device (see FIG. 1) which is considered to affect the recycling system, however, the operating conditions which can be contained are not limited thereto. It may suffice if the method for adjusting the concentration of starting materials of the present invention (I) is contained as one control method in the process.

The present invention (III) is described below. The present invention (III) is a process for producing a lower fatty acid from a lower olefin and oxygen in the presence of a catalyst, which is controlled by the method for controlling a process of the present invention (II).

According to the process for producing a lower fatty acid of the present invention (III), the method for controlling a process of the present invention (II) is contained as one control method in the process of producing a lower fatty acid from a lower olefin and oxygen in the presence of a catalyst, so that the process can be efficiently and stably controlled. Therefore, in the process for producing a lower fatty acid of the present invention (III), the site and method for measuring the starting material concentration and the method for controlling the feed amount of the newly added starting material based on the measured value are the same as those in the present invention (I) and of course, the concentration of starting materials is adjusted by the adjusting method of the present invention (I).

The catalyst which can be used in the process for producing a lower fatty acid of the present invention (III) is not particularly limited and any catalyst may be used as long as it has a capability of bringing about the oxidation of lower olefin with the oxygen to produce a lower fatty acid. Examples of the catalyst include a catalyst comprising palladium and a phosphoric acid or a sulfur-containing modifier (see, Japanese Unexamined Patent Publication Nos. 47-13221 and 51-29425 (JP-A-47-13221 and JP-A-51-29425)), a catalyst comprising a palladium salt of a certain kind of heteropolyacid (see, Japanese Unexamined Patent Publication No. 54-57488 (JP-A-54-57488)) and a catalyst comprising a Group 3-type oxygen compound (see, Japanese Unexamined Patent Publication No. 46-6763 (JP-A-46-6763)).

Among these, preferred is a catalyst comprising metal palladium-heteropolyacid and/or a salt thereof and specific examples thereof include the catalysts disclosed in Japanese Unexamined Patent Publications No. 7-89896 and No. 9-67298 (JP-A-7-89896 and JP-A-9-67298), however, the present invention is, of course, not limited thereto.

The catalyst may be a tablet of the catalyst component itself or may be a supported catalyst where the catalyst component is supported on a support. In the case of using a supported catalyst, the support which can be used is not particularly limited and a porous substance which can be usually used as the support can be used. Specific examples thereof include silica, diatomaceous earth, montmorillonite, titania, activated carbon, alumina and silica alumina, however, the present invention is not limited thereto.

The shape of the substance which can be used as a support of the catalyst for use in the present invention (III) is not particularly limited and specifically, substances having a powder, spherical, pellet or other arbitrary form may be used.

The support is preferably a support mainly comprising a siliceous substance and having a spherical or pellet form, more preferably a silica having a purity of 95% by mass or more based on the entire mass of the support.

The average particle size thereof is preferably from 2 to 10 mm in the case of a fixed bed, and from powder to 5 mm in the case of a fluidized bed.

In the process for producing a lower fatty acid of the present invention (III), the reaction temperature is not particularly limited. The optimal temperature varies depending on the kind of olefin as a starting material, the catalyst used and the like, however, in general, the reaction temperature is preferably from 100 to 300° C., more preferably from 120 to 250° C.

The reaction pressure is also not particularly limited. Similarly to the reaction temperature, the optimal value of course varies depending on the kind of olefin as a starting material, the catalyst used and the like. In general, the reaction pressure practically advantageous in view of the equipment is preferably from 0.0 to 3.0 MPa (gauge pressure), however, the present invention is not limited thereto. The reaction pressure is more preferably from 0.1 to 1.5 MPa (gauge pressure).

The starting material for use in the process for producing a lower fatty acid of the present invention (III) includes a lower olefin and oxygen.

The lower olefin is not particularly limited. One or more linear or branched olefin containing at least one unsaturated bond and having 6 or less carbon atoms is preferably used. More preferred examples thereof include, but are not limited to, ethylene, propylene, 1-butene, 2-butene, butadiene and/or a mixture thereof, with ethylene being still more preferred.

Within the range of not affecting the reaction, other compounds, for example, a lower saturated hydrocarbon such as methane, ethane and propane, may be mixed.

Oxygen is not particularly limited. Oxygen diluted with an inert gas such as nitrogen and carbon dioxide may be used and, needless to say, high-purity oxygen may be used, and for example, the oxygen may also be fed in the form of air. In general, oxygen having a high concentration, suitably having a purity of 99% or more, is advantageous.

The concentrations of the lower olefin and oxygen as starting materials in the gas are not particularly limited. Similarly to the reaction temperature and pressure, the optimal values of course vary depending on the kind of olefin as a starting material, the catalyst used and the like. In general, the lower olefin ratio is fed to the reaction system to occupy a ratio of 5 to 80 vol %, preferably from 8 to 50 vol %, and oxygen is added to occupy a ratio of 1 to 15 vol %, preferably from 3 to 10 vol %, more preferably from 4 to 8 vol %.

The gas hourly space velocity (hereinafter simply referred to as “GHSV”) is also not particularly limited. In general, the gas in the standard state is preferably passed through the catalyst at 10 to 10,000 Hr⁻¹, more preferably from 300 to 5,000 Hr⁻¹, but the present invention is not limited thereto.

Particularly, in the case of using a catalyst comprising metal palladium, and a heteropolyacid and/or a salt thereof, when water is allowed to be present within the reaction system, an extremely high effect is provided on the improvement of activity and selectivity of producing a lower fatty acid and on the maintenance of activity of the catalyst. The water vapor is suitably contained in the reaction gas in a ratio of 1 to 50 vol %, preferably from 5 to 40 vol %.

The present invention (IV) is described below. The present invention (IV) is a process for producing a lower fatty acid ester from a lower olefin and a lower fatty acid in the presence of a catalyst, which is controlled by the method for controlling a process of the present invention (II).

According to the process for producing a lower fatty acid ester of the present invention (IV), the method for controlling a process of the present invention (II) is contained as one control method, so that in the process of producing a lower fatty acid ester from a lower olefin and a lower fatty acid in the presence of a catalyst, the process can be efficiently and stably controlled. Therefore, in the process for producing a lower fatty acid ester of the present invention (IV), the site and method for measuring the starting material concentration and the method for controlling the feed amount of the newly added starting material based on the measured value are the same as those in the present invention (I) and of course, the concentration of starting materials is adjusted by the adjusting method of the present invention (I).

The catalyst which can be used in the process for producing a lower fatty acid ester of the present invention (IV) is not particularly limited. It is generally well known that when a lower olefin and a lower fatty acid are reacted in the presence of an acidic catalyst, a corresponding fatty acid ester can be obtained. In this reaction, a heteropolyacid and/or a salt thereof is known to act as an effective catalyst. Specific examples thereof include, but are of course not limited to, the catalysts disclosed in Japanese Unexamined Patent Publications No. 4-139148, No. 4-139149, No. 5-65248, No. 5-163200, No. 5-170699, No. 5-255185, No. 5-294894, No. 6-72951 and No. 9-118647 (JP-A-4-139148, JP-A-4-139149, JP-A-5-65248, JP-A-5-163200, JP-A-5-70699, JP-A-5-255185, JP-A-5-294894, JP-A-6-72951 and JP-A-9-118647).

The catalyst may be a tablet of the catalyst component itself or may be a supported catalyst where the catalyst component is supported on a support. In the case of using a supported catalyst, the support which can be used is not particularly limited and a porous substance which can be usually used as the support can be used. Specific examples thereof include silica, diatomaceous earth, montmorillonite, titania, activated carbon, alumina and silica alumina, however, the present invention is not limited thereto.

The shape of the substance which can be used as a support of the catalyst for use in the present invention (IV) is not particularly limited and specifically, substances having a powder, spherical, pellet or other arbitrary form may be used.

The support is preferably a support mainly comprising a siliceous substance and having a spherical or pellet form, more preferably a silica having a purity of 95% by mass or more based on the entire mass of the support.

The average particle size thereof is preferably from 2 to 10 mm in the case of a fixed bed, and from powder to 5 mm in the case of a fluidized bed.

In the process for producing a lower fatty acid ester of the present invention (IV), the reaction temperature and the reaction pressure are not particularly limited except that these must fall within the range of keeping the lower fatty acid used as a starting material in the gas state. Therefore, the reaction temperature and the reaction pressure duly vary according to the lower fatty acid used as a starting material. In general, the reaction temperature is preferably from 100 to 300° C., more preferably from 120 to 250° C.

Although the reaction pressure must be selected by taking account of the balance with the reaction temperature, in general, the reaction pressure practically advantageous in view of the equipment is preferably from 0.0 to 3.0 MPa (gauge pressure), more preferably from 0.1 to 1.5 MPa (gauge pressure), however, the present invention is not limited thereto.

The starting material for use in the process for producing a lower fatty acid ester of the present invention (IV) includes a lower olefin and a lower fatty acid.

The lower olefin is not particularly limited. One or more of linear or branched olefins containing at least one unsaturated bond and having 6 or less carbon atoms is preferably used. More preferred examples thereof include, but are not limited to, ethylene, propylene, 1-butene, 2-butene, butadiene and/or a mixture thereof, with ethylene being still more preferred.

Within the range of not affecting the reaction, other compounds, for example, a lower saturated hydrocarbon such as methane, ethane and propane, may be mixed.

The lower fatty acid is not particularly limited. A carboxylic acid having 4 or less carbon atoms is preferred but the present invention is not limited thereto. Specific examples thereof include formic acid, acetic acid, propionic acid, acrylic acid and methacrylic acid. Among these, acetic acid and acrylic acid are preferred.

With respect to the use ratio of a lower olefin and a lower fatty acid, the lower olefin is preferably used in a molar amount equal to or in excess of the lower fatty acid. The molar ratio of lower olefin:lower fatty acid is preferably from 1:1 to 30:1, more preferably from 10:1 to 20:1.

The GHSV is also not particularly limited. In general, the gas in the standard state is preferably passed through the catalyst at 10 to 10,000 Hr⁻¹, more preferably from 300 to 5,000 Hr⁻¹, but the present invention is not limited thereto.

Particularly, in the case of using a catalyst comprising a heteropolyacid and/or a salt thereof, a slight amount of water is preferably allowed to be present within the reaction system from the standpoint of the catalyst life. However, if an excessively large amount of water is added, by-products such as ethanol and diethyl ether also disadvantageously increase. In general, the amount of water used is preferably from 1 to 15 mol %, more preferably from 3 to 8 mol %, based on the entire amount of the lower olefin and the lower fatty acid used.

The present invention (V) is described below. The present invention (V) is a process for producing a lower fatty acid ester from a lower olefin, oxygen and a lower fatty acid in the presence of a catalyst, which is controlled by the method for controlling a process of the present invention (II).

According to the process for producing a lower fatty acid ester of the present invention (V), the method for controlling a process of the present invention (II) is contained as one control method, so that in the process of producing a lower fatty acid ester from a lower olefin, oxygen and a lower fatty acid in the presence of a catalyst, the process can be efficiently and stably controlled. Therefore, in the process for producing a lower fatty acid ester of the present invention (V), the site and method for measuring the starting material concentration and the method for controlling the feed amount of the newly added starting material based on the measured value are the same as those in the present invention (I) and of course, the concentration of starting materials is adjusted by the adjusting method of the present invention (I).

The catalyst which can be used in the process for producing a lower fatty acid ester of the present invention (V) is not particularly limited and any catalyst may be used as long as it has a capability of producing a lower fatty acid ester from a lower olefin, a lower fatty acid and oxygen. Examples of the catalyst include those where palladium is used as a main component, an alkali metal or alkaline earth metal and at least on metal such as gold, copper, molybdenum, cadmium, lead, vanadium, bismuth, chromium, tungsten, manganese or iron are used as co-catalysts, and these catalyst components are supported usually on alumina, silica, activated carbon or pumice titanium oxide. Specific examples thereof include the catalysts disclosed in Japanese Unexamined Patent Publications No. 2-91045, No. 5-186393, No. 6-47281, No. 7-136515, No. 7-308576 and No. 8-38899 (JP-A-2-91045, JP-A-5-186393, JP-A-6-47281, JP-A-7-136515, JP-A-7-308576 and JP-A-8-38899), however, the present invention is of course not limited thereto.

The catalyst may be a tablet of the catalyst component itself or may be a supported catalyst where the catalyst component is supported on a support. In the case of using a supported catalyst, the support which can be used is not particularly limited and a porous substance which can be usually used as the support can be used. Specific examples thereof include silica, diatomaceous earth, montmorillonite, titania, activated carbon, alumina and silica alumina, however, the present invention is not limited thereto.

The shape of the substance which can be used as a support of the catalyst for use in the present invention (V) is not particularly limited and specifically, substances having a powder, spherical, pellet or other arbitrary form may be used.

The support is preferably a support mainly comprising a siliceous substance and having a spherical or pellet form, more preferably a silica having a purity of 90% by mass or more based on the entire mass of the support.

The average particle size thereof is preferably from 2 to 10 mm in the case of a fixed bed, and from powder to 5 mm in the case of a fluidized bed.

In the process for producing a lower fatty acid ester of the present invention (V), the reaction temperature is not particularly limited. The optimal temperature varies depending on the kind of olefin as a starting material, the catalyst used and the like, and it may suffice if the reaction temperature is in the range of keeping the starting material lower fatty acid in the gas state. In general, the reaction temperature is preferably from 100 to 300° C., more preferably from 120 to 250° C.

The reaction pressure is also not particularly limited. Similarly to the reaction temperature, the optimal value of course varies depending on the kind of olefin as a starting material, the catalyst used and the like. In general, the reaction pressure practically advantageous in view of the equipment is preferably from 0.0 to 3.0 MPa (gauge pressure), however, the present invention is not limited thereto. The reaction pressure is more preferably from 0.1 to 1.5 MPa (gauge pressure).

The starting material for use in the process for producing a lower fatty acid ester of the present invention (V) includes a lower olefin, a lower fatty acid and oxygen.

The lower olefin is not particularly limited. One or more of linear or branched olefins containing at least one unsaturated bond and having 6 or less carbon atoms is preferably used. More preferred examples thereof include, but are not limited to, ethylene, propylene, 1-butene, 2-butene, butadiene and/or a mixture thereof, with ethylene and propylene being still more preferred.

Within the range of not affecting the reaction, other compounds, for example, a lower saturated hydrocarbon such as methane, ethane and propane, may be mixed.

The lower fatty acid is not particularly limited. A carboxylic acid having 4 or less carbon atoms is preferred but the present invention is not limited thereto. Specific examples thereof include formic acid, acetic acid, propionic acid, acrylic acid and methacrylic acid. Among these, acetic acid and acrylic acid are preferred.

Oxygen is not particularly limited. Oxygen diluted with an inert gas such as nitrogen and carbon dioxide may be used and, needless to say, high-purity oxygen, and for example, the oxygen may also be fed in the form of air. In general, oxygen having a high concentration, suitably having a purity of 99% or more, is advantageous.

The concentrations of the lower olefin and oxygen as starting materials in the gas are not particularly limited. Similarly to the reaction temperature and pressure, the optimal values of course vary depending on the kind of olefin as a starting material, the catalyst used and the like. In general, the lower olefin ratio is fed to the reaction system to occupy a ratio of 5 to 80 vol %, preferably from 8 to 60 vol %, and oxygen is added to occupy a ratio of 1 to 15 vol %, preferably from 3 to 10 vol %, most preferably from 4 to 8 vol %.

To speak specifically about the process for producing a lower fatty acid ester of the present invention (V), in the case of a process for producing allyl acetate using acetic acid as the lower fatty acid and propylene as the lower olefin, the propylene is preferably used in an amount giving a concentration ratio of 40 to 60 vol %. In the case of a process for producing vinyl acetate using acetic acid as the lower fatty acid and ethylene as the lower olefin, the ethylene is preferably fed in an amount of giving a concentration ratio of 20 to 40 vol %.

With respect to the use ratio of a lower olefin and a lower fatty acid, the lower olefin is preferably used in a molar amount equal to or in excess of the lower fatty acid. The molar ratio of lower olefin:lower fatty acid is preferably from 1:1 to 30:1, more preferably from 2:1 to 10:1.

The GHSV is also not particularly limited. In general, the gas in the standard state is preferably passed through the catalyst at 10 to 10,000 Hr⁻¹, more preferably from 300 to 5,000 Hr⁻¹, but the present invention is not limited thereto.

The present invention is described in greater detail below by referring to the Examples and Comparative Examples, however, these Examples are only to show the outline of the present invention, and the present invention should not be construed as being limited to these Examples.

The present invention is described by referring to a reaction process of obtaining acetic acid from ethylene and oxygen in the presence of a heteropolyacid-based catalyst for the production of acetic acid.

Outline of Reaction

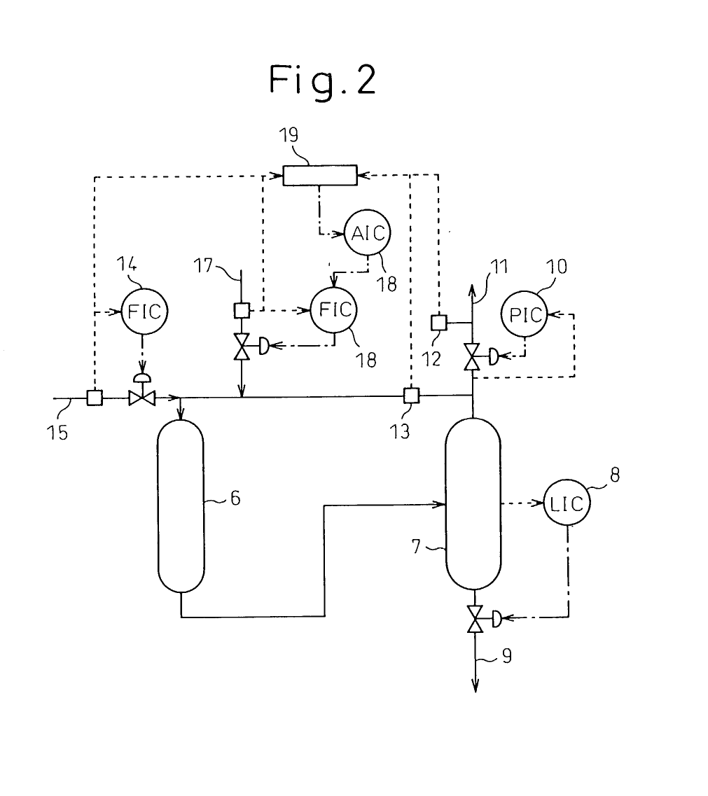

FIG. 2 shows a reaction process constructed to have a recycling system of feeding ethylene and oxygen as starting materials to a reactor, separating acetic acid through a gas-liquid separation device and returning the gas containing inert and unreacted starting materials to the reactor. The main product is acetic acid and as by-products, carbon dioxide, acetaldehyde, ethanol, methyl acetate, propionic acid and the like are produced.

The target conditions in the reactor were such that the reaction peak temperature of the catalyst bed was 200° C., the reaction pressure was 0.75 MPa (gauge pressure) and the GHSV was 1,800 Hr⁻¹.

Preparation Process of Catalyst

A catalyst for the production of acetic acid used in Examples 1 and 2 and Comparative Examples 1 and 2 is described below.

2.81 g of sodium tetrachloropalladate, 1.05 g of chloroauric acid and 0.1402 g of zinc chloride were weighed and therein, pure water was dissolved to make 45 ml, thereby preparing Aqueous Solution (1). To the beaker in which Aqueous Solution (1) was prepared, 69.6 g of a silica support (Support KA-1, produced by Sud Chemie, 5 mmΦ) was added and allowed to absorb the entire amount of Aqueous Solution (1).

In a separate beaker, 8.00 g of sodium metasilicate was weighed and thereto, 100 g of pure water was added and dissolved to prepare Aqueous Solution (2). The silica support having absorbed thereto Aqueous Solution (1) was added to the beaker in which Aqueous Solution (2) was prepared, and left standing at room temperature for 20 hours. Subsequently, 8.00 g of hydrazine monohydrate was gradually added thereto while stirring at room temperature. Thereafter, the catalyst was collected by filtration, washed by passing pure water therethrough, and dried at 110° C. for 4 hours in an air stream.

Then, 0.266 g of sodium tellurite was weighed and thereto 45 g of pure water was added to prepare Aqueous Solution (3). The metal palladium-supported catalyst prepared above was added to Aqueous Solution (3) and allowed to absorb the entire amount of Aqueous Solution (3). Thereafter, the catalyst was dried at 110° C. for 4 hours in an air stream to obtain a tellurium added metal palladium-supported catalyst.

In another separate beaker, 23.98 g of tungstosilicic acid hexacosahydrate was weighed and thereto, pure water was added to make 45 ml, thereby preparing Aqueous Solution (4). The metal palladium-supported catalyst washed with an acidic solution was added to the beaker in which Aqueous Solution (4) was prepared and allowed to absorb the entire amount of Aqueous Solution (4). Thereafter, the catalyst was dried at 110° C. for 4 hours in an air stream to obtain a catalyst for the production of acetic acid.

By such an operation, the catalyst was prepared in an amount of about 5 1 which is an amount necessary for the filling a reactor.

Outline of Process

The outline of the process used in the Examples and Comparative Examples is described below by referring to FIG. 2.

The reactor (6) used was a vertical tubular reactor with a jacket. By making use of the vaporization of water fed to the jacket, the heat generated due to the reaction was eliminated and thereby the temperature within the reactor was controlled.

In the gas-liquid separation device (7), the gas fed in the gas-liquid mixed state and passed through the reactor is separated into a gas mainly comprising unreacted ethylene gas and containing unreacted oxygen, carbon dioxide, nitrogen and the like, and a liquid comprising acetic acid, water and the like. The separated liquid containing acetic acid is taken out from the system through an acetic acid discharge line (9). On the other hand, although a part of the gas is released out of the process through a purge line (11), most of the gas is returned to the reactor through a flow meter (13) after adding thereto fresh ethylene from an ethylene feed line (17) and oxygen from an oxygen feed line (15).

The process, particularly the manipulate variable (MV) in the feed amounts of starting materials, is automatically controlled as follows. The starting material concentration at the inlet of the reactor (6) is computed by a computing device (19) based on the measured values by respective control devices and measuring devices, and the computed value is input as a process variable of the concentration controller (18). In the concentration controller (18), a new flow rate set-point value given to the flow rate control device (16) is calculated based on the deviation between a preliminarily given set-point value (SV) and the PV above. As a result, a construction of so-called concentration-flow rate cascade control by PID controller is established and thereby MV is decided.

The control valves used for the pressure control device (10) and the ethylene flow rate control device (16) both are a VSM-type control valve manufactured by Yamatake, having a CV value of 0.05 and having linear characteristics (LC), and the control valves are each connected to a ½ inch pipeline.

### EXAMPLE 1

In the process where the control line was constructed as shown in FIG. 2, the starting material gas composition at the reactor inlet was adjusted to give a volume ratio (vol %) of oxygen:ethylene:water:nitrogen=4.5:8.5:25:62, and a concentration-flow rate cascade control was performed.

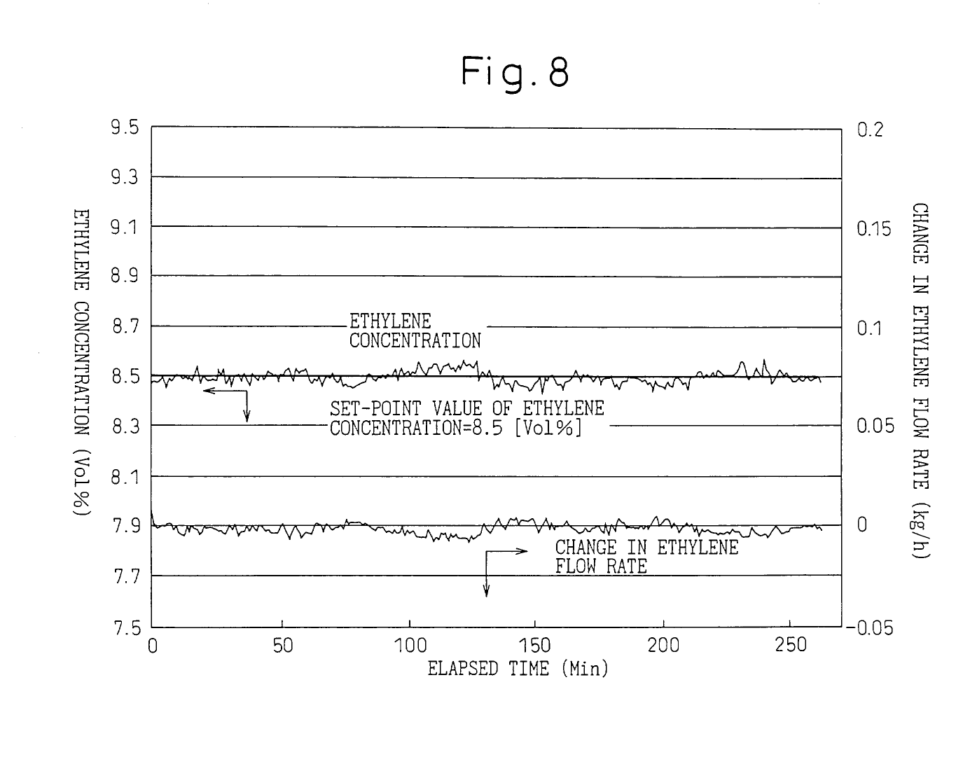

As a result, the process exhibited very stable ethylene concentration behavior as shown in FIG. 8.

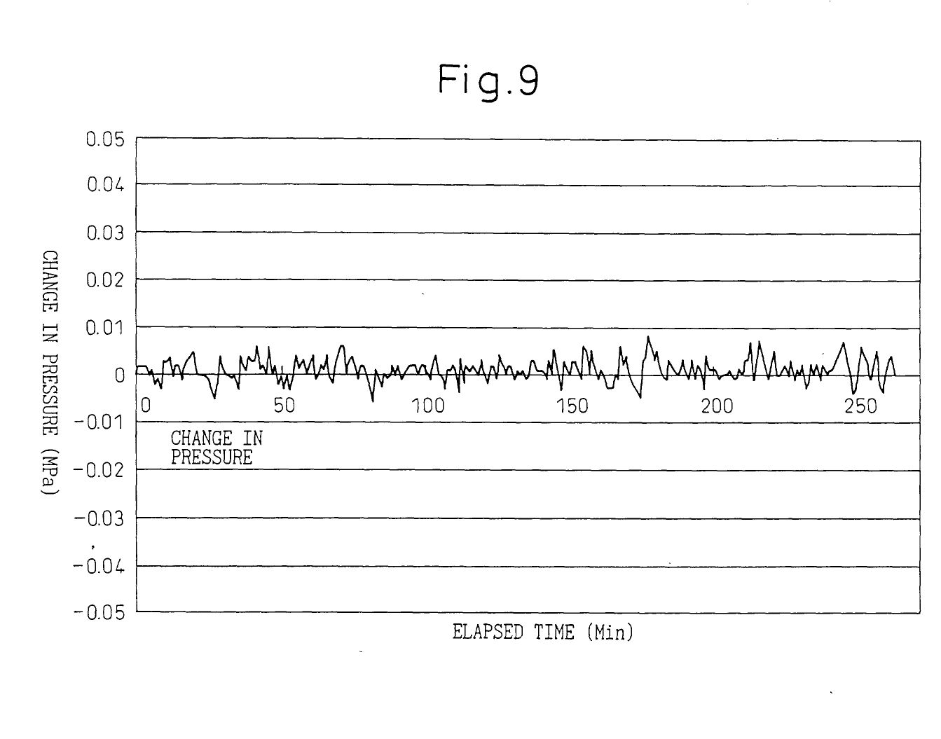

The sum of squares deviation as an index of showing the control results, namely, the area size obtained by squaring the deviation from the target ethylene concentration, was 0.187 (vol %)² and thus, very small. At this time, the pressure behavior was very stable as shown in FIG. 9 and the square deviation of the pressure was 0.00182 MPa².

The PID parameters of the ethylene concentration controller in the primary side of the concentration-flow rate cascade control were a proportional band of 200% and an integral time of 600 seconds.

### EXAMPLE 2

In the process where the control line was constructed as shown in FIG. 2, the starting material gas composition at the reactor inlet was adjusted to give volume ratio (vol %) of oxygen:ethylene:water:nitrogen=4.5:8.5:25:62, a concentration-flow rate cascade control was performed; and after 30 minutes, an operation of changing the set-point value (SV) of the ethylene concentration from 8.5 vol % to 9.0 vol % was added.

Despite the addition of this operation, in the unit of the present invention, the set-point value could be reached within about 1 hour while allowing the process to proceed very stably without causing any overshoot or unstable action. At this time, the square deviation from the set-point value of the ethylene concentration was 6.48 (vol %)² and the square deviation of the pressure was 0.00175 MPa².

The PID parameters of the ethylene concentration controller were similarly a proportional band of 200% and an integral time of 600 seconds.

### COMPARATIVE EXAMPLE 1

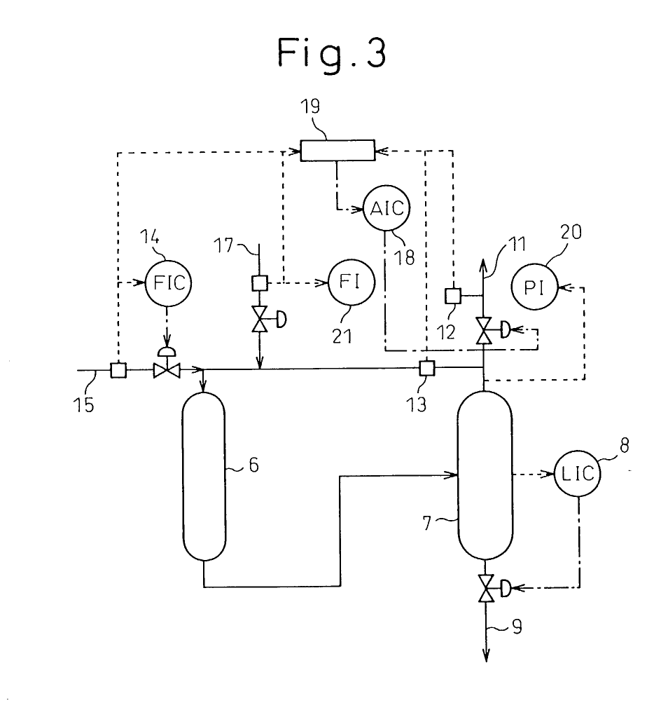

As shown in FIG. 3, a reaction process of operating the purge flow rate to give a constant ethylene concentration was constructed.

In FIG. 3, although the process was the same as in Examples 1 and 2, the operation signal MV of the concentration controller (18) was connected to switch the control valve of the purge line (11) and the control valve of the ethylene feed line (17) was fixed at an opening of 17.7% (full close at 0%).

In this process, the reactor inlet composition was adjusted to give a volume ratio (vol %) of oxygen:ethylene:water:nitrogen=4.5:8.5:25:60.5, then the concentration control was performed, and after 30 minutes, an operation of changing the set-point value (SV) of the ethylene concentration from 8.5 vol % to 9.0 vol % was added.

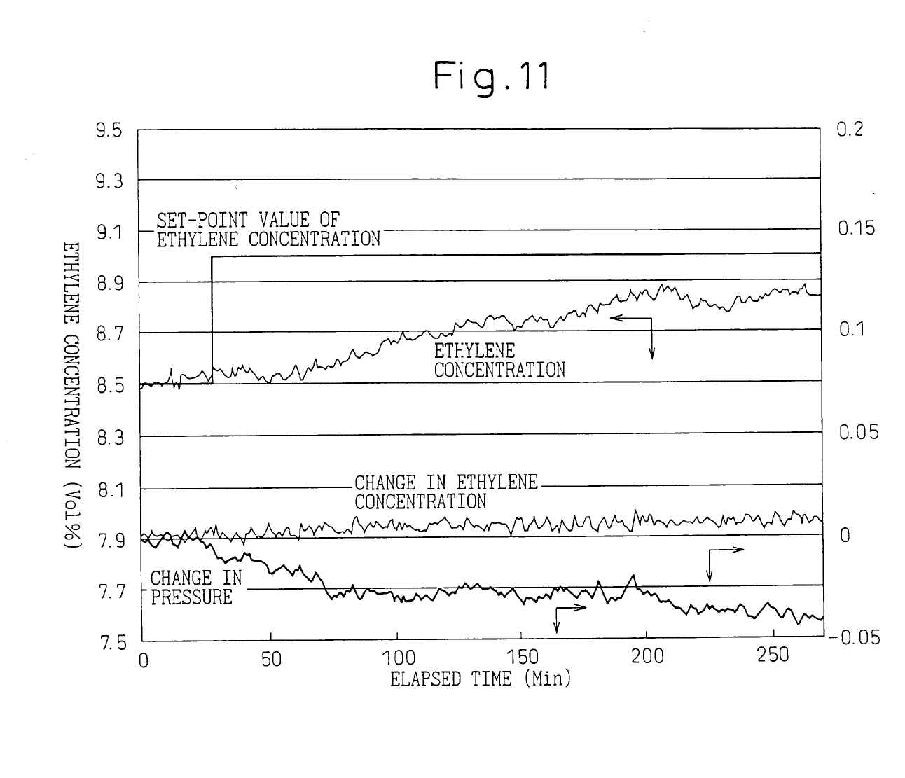

As seen from the results shown in FIG. 11, by performing an operation of increasing the purge flow rate and accelerating the inflow of ethylene into the system to elevate the ethylene concentration in the concentration controller (18), the pressure was greatly reduced, however, the increase in the ethylene flow rate resulting from the pressure reduction was not sufficiently large to reach the set-point value of the ethylene concentration and since, even after 4 hours, the set-point value was not reached, the control had to be given up.

At this time, the square deviations of the ethylene concentration and the pressure were 22.83 (vol %)² and 0.203 MPa², respectively, and despite the disturbance in the pressure as large as about 100 times as compared with Example 2, the control result of the ethylene concentration was very bad and even the set-point value could not be reached.

By taking account of the effect on the ethylene concentration and the disturbance in the pressure, the PID parameters of the ethylene concentration controller were a proportional band of 60% and an integral time of 1,200 seconds.

### COMPARATIVE EXAMPLE 2

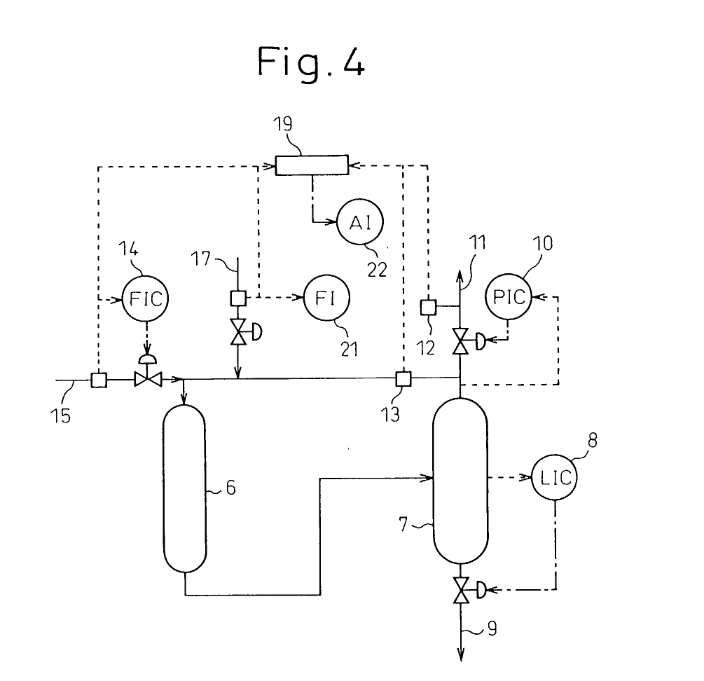

As shown in FIG. 4, a reaction process of controlling the purge amount to give a constant pressure was constructed. In FIG. 4, although the process was the same as in Examples 1 and 2, the control valve of the ethylene feed line (17) was fixed at an opening of 17.4% (full admission at 0%).

In this process, the reactor inlet composition was adjusted to give a volume ratio (vol %) of oxygen:ethylene:water:nitrogen=4.5:8.5:25:62, and then the pressure control was performed.

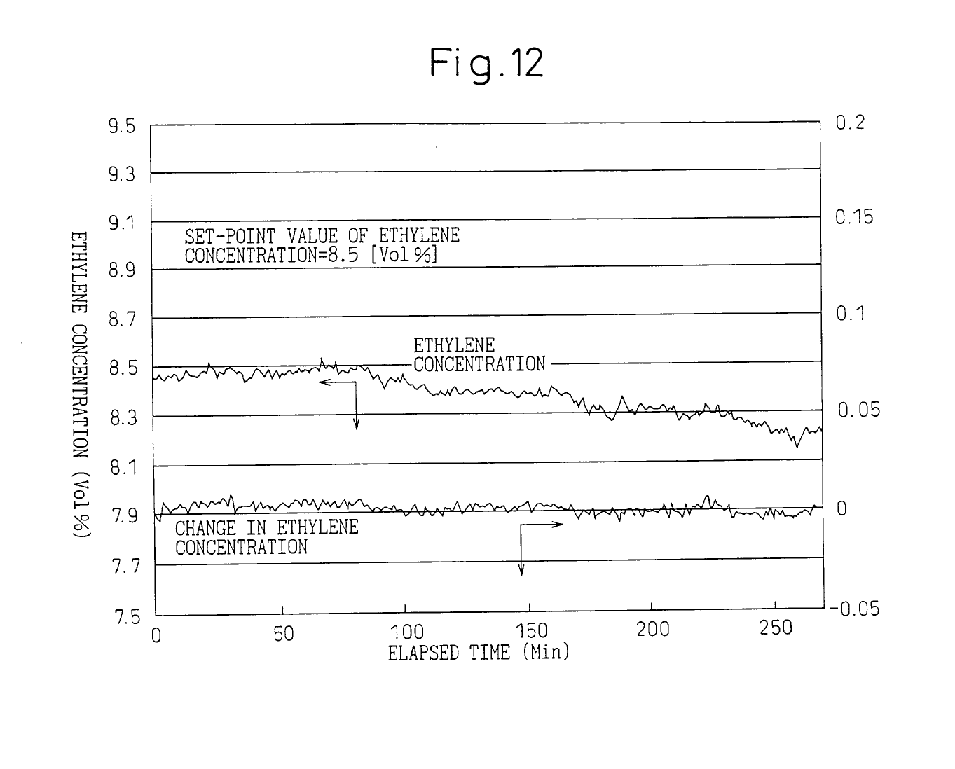

As seen from the results shown in FIG. 12, due to the effect by the fluctuation in the purge amount changed in the pressure control device (10) to eliminate the deviation of the pressure, the ethylene flow rate slightly varied and since the ethylene concentration gradually reduced and even after 4 hours, the set-point value was not recovered, the control was given up.

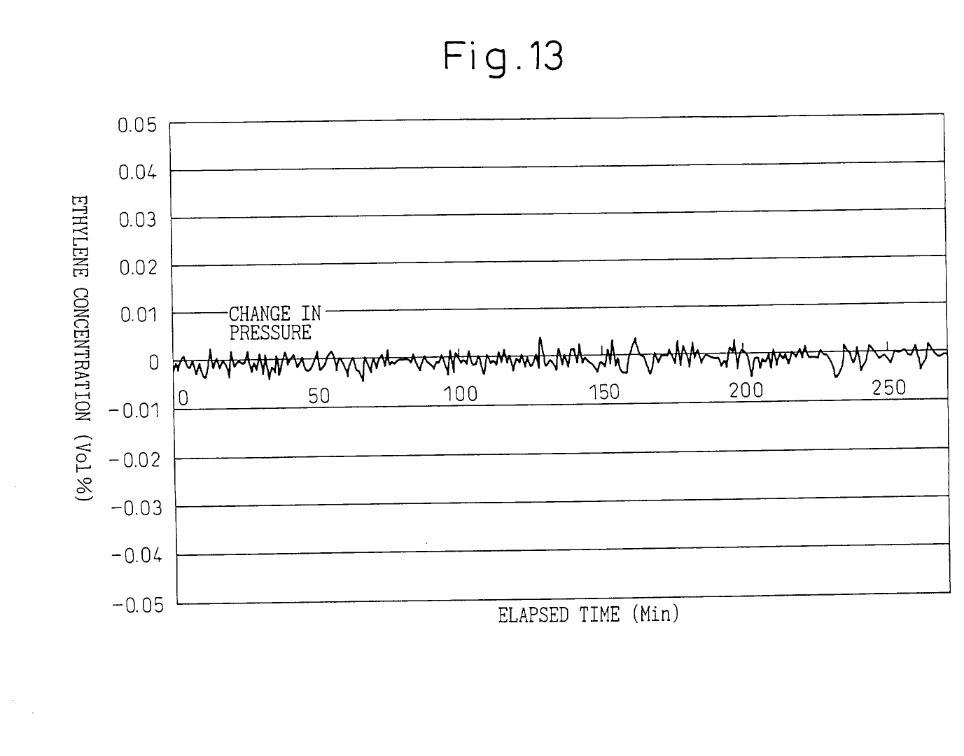

At this time, the square deviation of the ethylene concentration was 6.09 (vol %)² and as high as 30 times that of Example 1, revealing that the effect of keeping the ethylene concentration at the set-point value was not provided. FIG. 13 shows the pressure behavior at this time. The square deviation of the pressure was 0.00108 MPa² and this control result was on the same level as in Example 1.

### EXAMPLE 3

The present invention is described below by referring to a reaction process of obtaining ethyl acetate from ethylene and acetic acid in the presence of a heteropolyacid-based catalyst for the production of ethyl acetate.

Outline of Reaction and Process

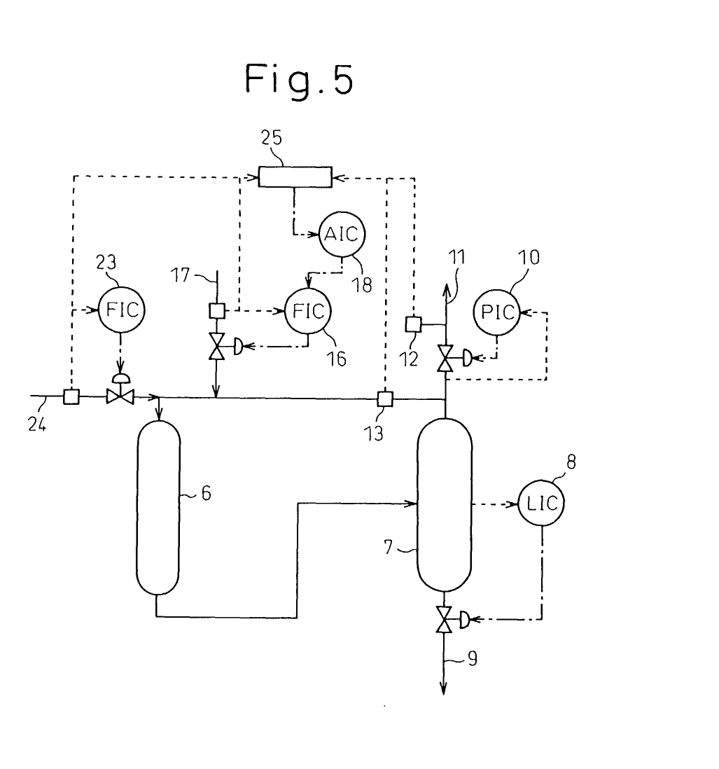

As shown in FIG. 5, a process was constructed to have a recycling system of feeding ethylene and acetic acid as starting materials to a reactor, separating ethyl acetate through a gas-liquid separation device and returning the gas containing inert and unreacted starting materials to the reactor. FIG. 5 shows the same process as in FIG. 2 except that the line for feeding oxygen in FIG. 2 was used here as an acetic acid feed line (24) and an acetic acid flow rate control device (23) and in the computing device (25), the program was changed to calculate the ethylene concentration from the acetic acid flow rate in place of the oxygen flow rate.

Preparation Process of Catalyst

A natural silica (KA-0, produced by Sud Chemie) was used as a support and this was previously dried in a hot air dryer adjusted to 110° C. for 4 hours. 3,266.5 g of tungstophosphoric acid (H₃PW₁₂O₄₀) and 6.6 g of lithium nitrate were weighed and thereto, 750 ml of pure water was added and dissolved to obtain an aqueous Li_{0.1}H_{2.9}PW₁₂O₄₀ solution. This aqueous solution was then diluted with pure water to make 1,700 ml and uniformly stirred. Subsequently, 2,790 g of the support dried above was weighed, immersed in the aqueous solution, and fully impregnated with the aqueous solution with thorough stirring. The support impregnated with the solution was air dried for 1 hour and then dried in a hot air dryer adjusted to 150° C. for 5 hours. The thus-obtained catalyst had a weight of 5,675 g.

About 5 1 of the catalyst obtained through the above-described procedure was filled in a reactor shown in FIG. 5, the conditions were adjusted such that the reaction peak temperature of the catalyst bed was 170° C., the reaction pressure was 0.8 MPa (gauge pressure), the space velocity (GHSV) was 1,500 Hr⁻¹, and the mixed gas fed to the reactor had a ratio of ethylene:acetic acid:water:nitrogen=78.5:8.0:4.5:9.0, and then the concentration-flow rate cascade control was performed.

After the passing of 30 minutes, an operation of elevating the set-point value (SV) of the starting material ethylene concentration by 0.5 vol % from 78.5 vol % was performed.

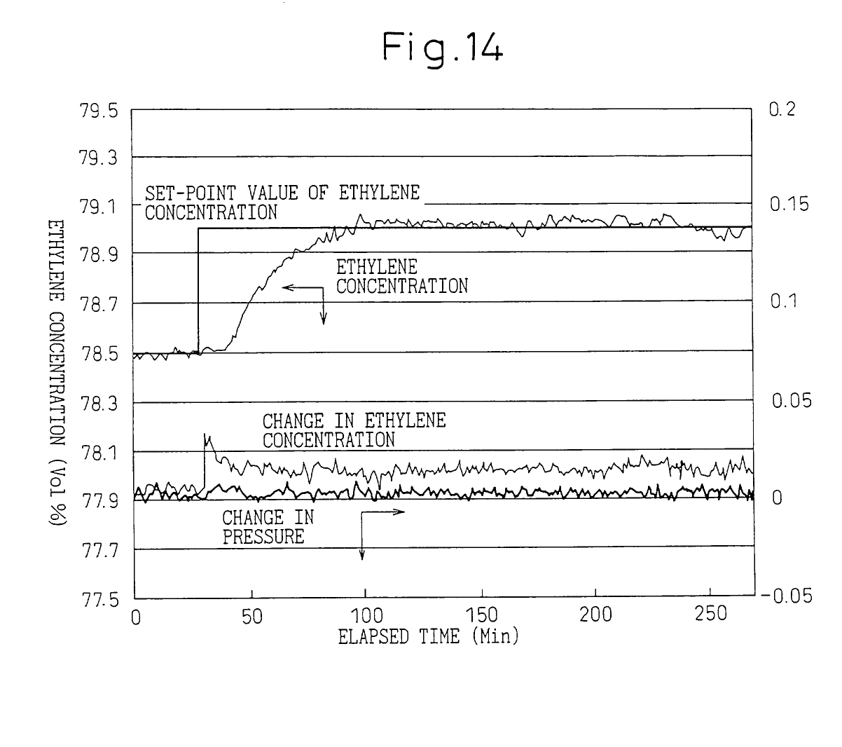

As seen from the results shown in FIG. 14, while showing stable behavior, the target ethylene concentration settled after about 2 hours. The square deviations of the ethylene concentration and the pressure were 5.54 (vol %)² and 0.00136 MPa², respectively.

### EXAMPLE 4

The present invention is described below by referring to a process of producing ally acetate from propylene, acetic acid and oxygen in the gas phase.

Outline of Reaction and Process

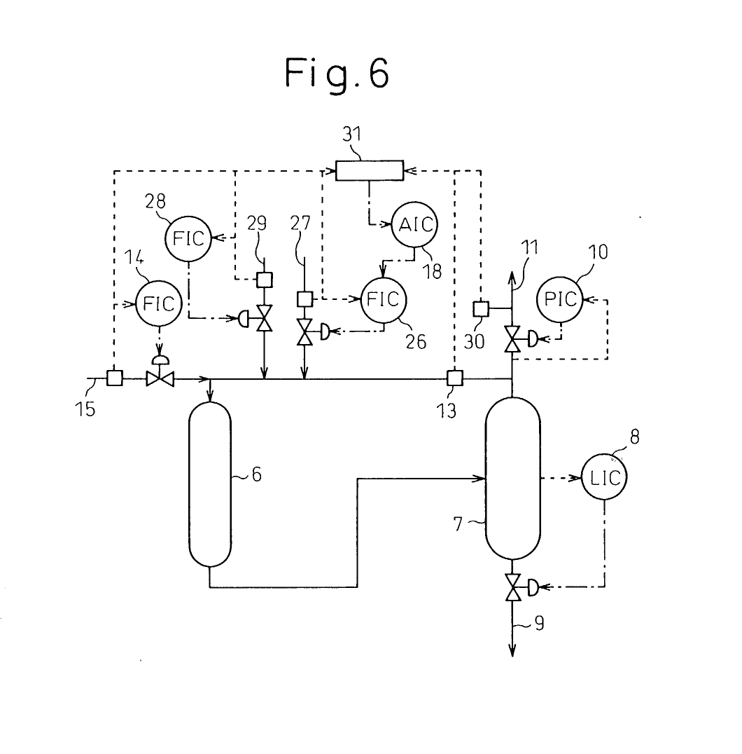

As shown in FIG. 6, a process of producing allyl acetate from starting materials of propylene, acetic acid and oxygen was constructed.

FIG. 6 shows the same process as in FIG. 2 except that the line used as an ethylene feed line in FIG. 2 was used here as a propylene feed line (27) and a propylene flow rate control device (26), and an acetic acid feed line (29) and an acetic acid flow rate control device (28) were newly added and operated.

The ethylene concentration analyzer in FIG. 2 was replaced by a propylene concentration analyzer (30) and the computing device (31) was programmed to calculate the propylene concentration from the flow rates of those starting materials.

Preparation Process of Catalyst

To 1,800 ml of an aqueous solution containing 45.6 g of sodium tetrachloropalladate (Na₂PdCl₄) and 5.2 g of copper chloride (CuCl₂), 5 1 of silica support having a particle size of 5 mm was added and impregnated with the entire amount of the aqueous solution. This catalyst was added to 4 1 of an aqueous solution containing 29.6 g of sodium hydroxide (NaOH), then alkali-treated at room temperature for 20 hours, and reduction-treated by adding thereto hydrazine hydrate. After the reduction, the catalyst was washed with water until chloride ions were not recognized, then dried at 110° C. for 4 hours, charged into 1,800 ml of an aqueous solution containing 150 g of potassium acetate (KOAc), allowed to absorb the entire solution, and again dried at 100° C. for 20 hours.

5 1 of the thus-prepared catalyst was filled in a stainless steel-made reaction tube shown in FIG. 6 and the reaction was performed by feeding a mixed gas containing 30% of propylene, 7.0% of acetic acid, 7.0% of oxygen, 14.0% of water and 42.0% of nitrogen at GHSV of 2,100 Hr⁻¹ and adjusting the conditions such that the reaction temperature was 165° C. and the pressure was 0.5 MPa (gauge pressure), and then the concentration-flow rate cascade control was performed. After 30 minutes, the propylene concentration SV was elevated from 30 vol % to 30.5 vol %.

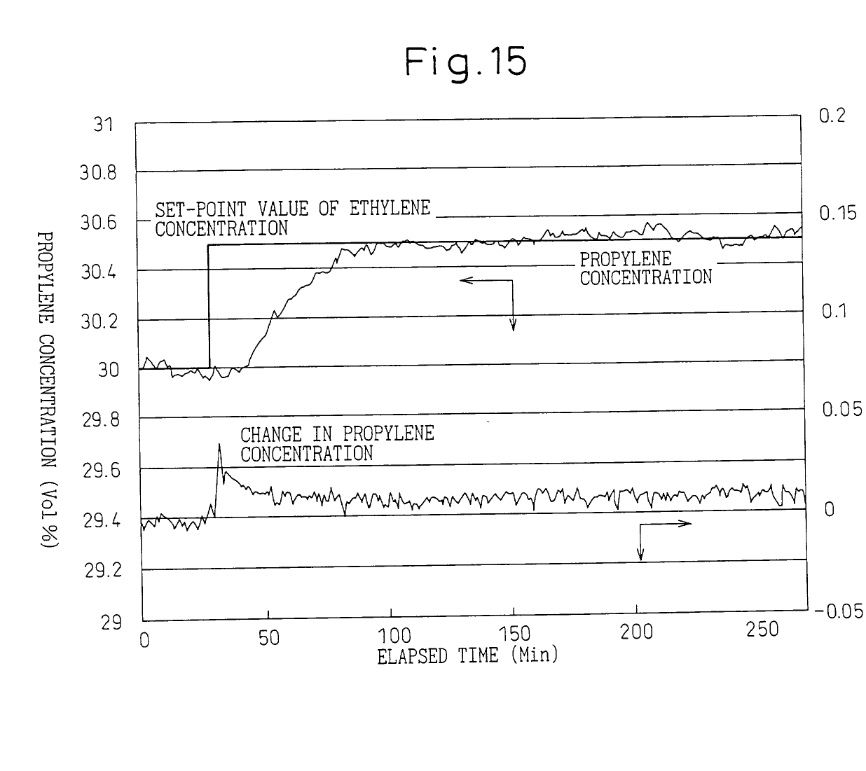

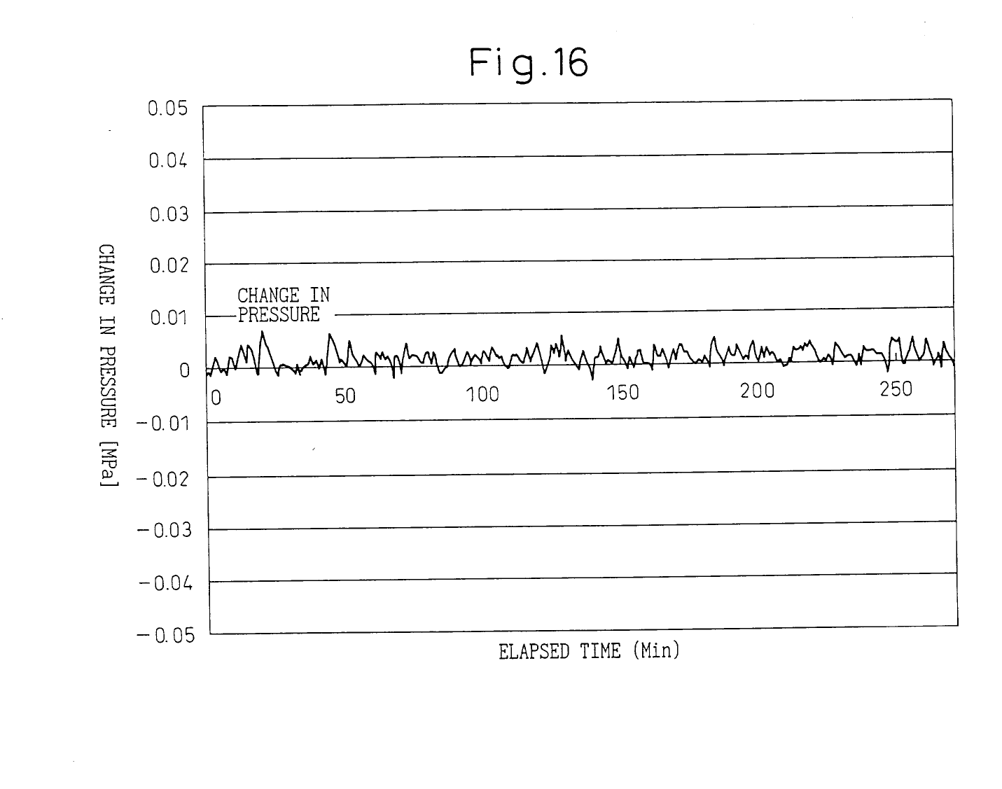

As seen from the results shown in FIGS. 15 and 16, while showing stable behavior, the target ethylene concentration settled after about 2 hours. The square deviations of the propylene concentration and the pressure were 6.76 (vol %)² and 0.00127 MPa², respectively.

### EXAMPLE 5

The present invention is described below by referring to a process of producing vinyl acetate in the presence of ethylene, acetic acid and oxygen using a palladium-based catalyst.

Outline of Reaction and Process

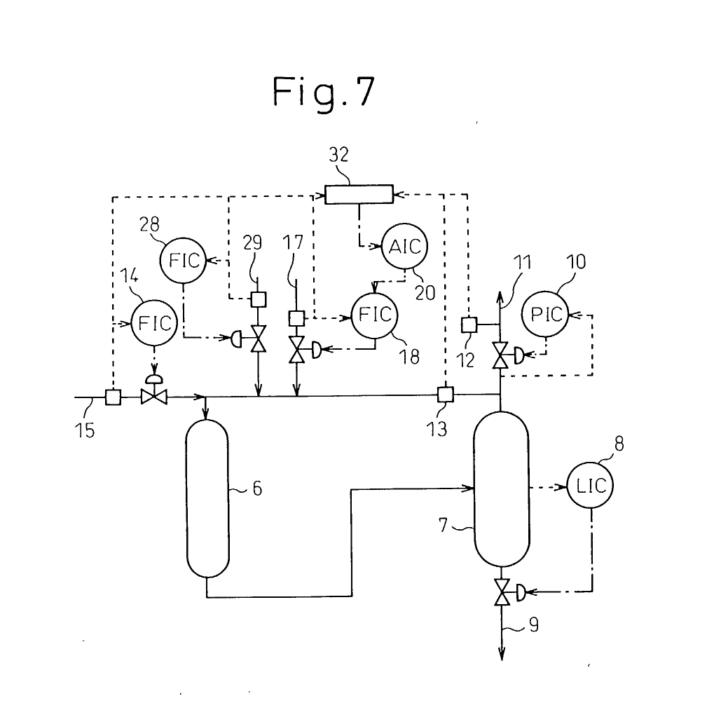

As shown in FIG. 7, a process of producing vinyl acetate from starting materials of ethylene, acetic acid and oxygen was constructed.

FIG. 7 shows the same process as in FIG. 2 except that, similarly to FIG. 6, an acetic acid feed line (29) and an acetic acid flow rate control device (28) were added, and the computing device (32) was programmed to calculate the ethylene concentration from the flow rates of those starting materials.

Preparation Process of Catalyst

In an aqueous solution containing 100 g of sodium tetrachloropalladate (Na₂PdCl₄) and 13 g of tetrachloroauric acid tetrahydrate, 5 1 of natural silica (KA-0, produced by Sud Chemie AG) was immersed and impregnated with the entire amount of the aqueous solution. This catalyst was added to 4 1 of an aqueous solution containing 320 g of sodium metasilicate and then left standing for 20 hours. Thereafter, sodium tetrachloropalladate and tetrachloroauric acid tetrahydrate were each reduced to a metal, washed with water and then dried at 110° C. for 4 hours. This support containing metal palladium and gold was then charged into an aqueous solution containing 4.0 g of tin acetate, allowed to absorb the entire solution, and dried at 110° C. for 4 hours. Subsequently, this catalyst containing metal palladium was charged into an aqueous solution containing 165 g of potassium acetate, allowed to absorb the entire solution, and dried at 110° C. for 4 hours.

5 1 of the thus-prepared catalyst was filled in a reaction tube shown in FIG. 7 and the reaction was performed by introducing a mixed gas prepared to a ratio of oxygen:acetic acid:water:nitrogen=60:4:15:15:6 at GHSV of 2,200 Hr⁻¹ under the conditions such that the reaction temperature was 170° C. and the pressure was 0.8 MPa (gauge pressure). In this process, the concentration-flow rate cascade control was performed and after 30 minutes, the ethylene concentration SV was elevated from 60 vol % to 60.5 vol %.

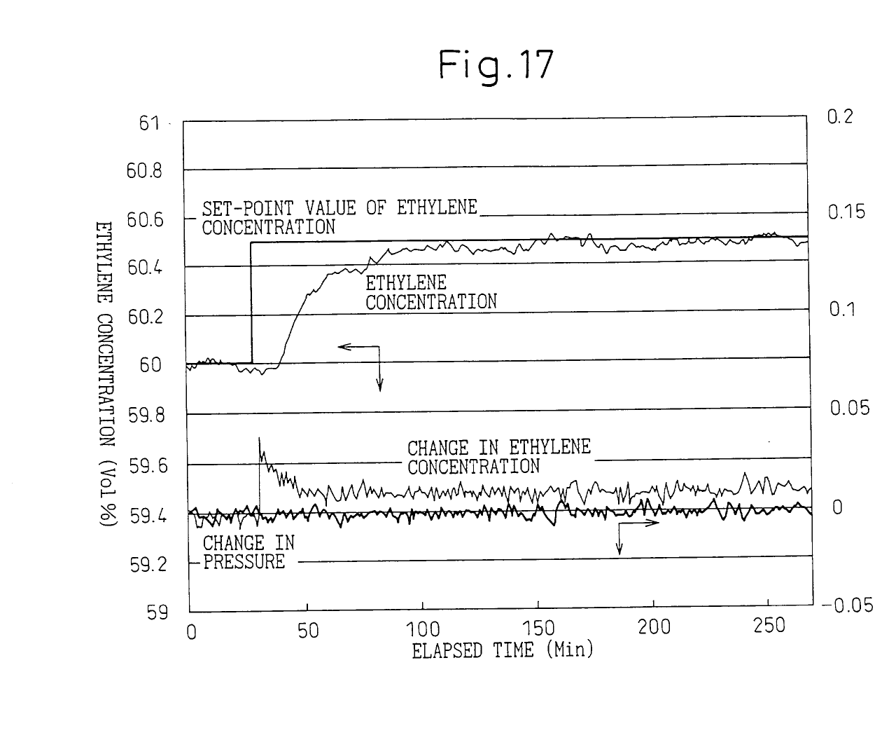

As seen from the results shown in FIG. 17, while showing stable behavior, the target ethylene concentration settled after about one and a half hours. The square deviations of the ethylene concentration and the pressure were 5.57 (vol %)² and 0.00101 MPa², respectively.

Industrial Applicability

As described in the foregoing pages, when the adjusting method of the present invention for adjusting the starting material concentration in a gas phase contact reaction and the process control method using the adjusting method are used, the reaction can be efficiently and stably performed.

Particularly, in the process for producing a lower fatty acid and the process for producing a lower fatty acid ester, using the method for adjusting the starting material concentration in a gas phase contact ration and the process control method by the adjusting method of the present invention, the reaction can be efficiently and stably over a long period of time as compared with those using a conventional process control method.

1. A method for adjusting the concentration of starting materials in a gas fed to a reactor in a gas phase contact reaction process having a recycling system, which comprises measuring the concentration of a starting material in a gas in said process, feeding the starting material by setting the feed amount of the starting material newly added to said process based on the measured value, and thereby controlling the concentration of the starting material in a gas fed to the reactor.

2. The method as claimed in claim 1, wherein the gas phase contact reaction is a fixed bed gas phase contact reaction.

3. The method as claimed in claim 1 or 2, wherein the reactor is a multitubular reactor and/or a multilayer reactor.

4. The method as claimed in any one of claims 1 to 3, wherein the site of measuring the concentration of a starting material is immediately before the reactor.

5. The method as claimed in any one of claims 1 to 4, wherein the feed amount of the starting material newly added is controlled by control means provided in the starting material feed line.

6. A method for controlling a reaction process, comprising controlling a gas phase contact reaction process, wherein at least one control method is the method for adjusting the concentration of starting materials as described in any one of claims 1 to 5.

7. A process for producing a lower fatty acid, comprising producing a lower fatty acid from a lower olefin and oxygen in the presence of a catalyst, wherein the method for adjusting the concentration of lower olefin and/or oxygen in the reactor is the adjusting method as described in any one of claims 1 to 5.

8. A process for producing a lower fatty acid, comprising producing a lower fatty acid from a lower olefin and oxygen in the presence of a catalyst, wherein the production process is controlled by the method for controlling a reaction process as described in claim 6.

9. The process as claimed in claim 7 or 8, wherein in the process for producing a lower fatty acid from a lower olefin and oxygen in the presence of a catalyst, the reaction is performed in the presence of water.

10. The process as claimed in any one of claims 7 to 9, wherein the lower olefin is at least one member selected from the group consisting of ethylene, propylene, 1-butene, 2-butene and butadiene.

11. A process for producing a lower fatty acid ester, comprising producing a lower fatty acid ester from a lower olefin and a lower fatty acid in the presence of a catalyst, wherein the method for adjusting the concentration of lower olefin and/or lower fatty acid in the reactor is the adjusting method as described in any one of claims 1 to 5.

12. A process for producing a lower fatty acid ester, comprising producing a lower fatty acid ester from a lower olefin and a lower fatty acid in the presence of a catalyst, wherein the production process is controlled by the method for controlling a reaction process as described in claim 6.

13. The process as claimed in claim 11 or 12, wherein in the process for producing a lower fatty acid ester from a lower olefin and a lower fatty acid in the presence of a catalyst, the reaction is performed in the presence of water.

14. A process for producing a lower fatty acid ester, comprising producing a lower fatty acid ester from a lower olefin, oxygen and a lower fatty acid in the presence of a catalyst, wherein the concentration of lower olefin, oxygen and/or lower fatty acid is adjusted by the adjusting method as described in any one of claims 1 to 5.

15. A process for producing a lower fatty acid ester, comprising producing a lower fatty acid ester from a lower olefin, oxygen and a lower fatty acid in the presence of a catalyst, wherein the production process is controlled by the method for controlling a reaction process as described in claim 6.

16. The process as claimed in claim 14 or 15, wherein in the process for producing a lower fatty acid ester from a lower olefin, oxygen and a lower carboxylic acid in the presence of a catalyst, the reaction is performed in the presence of water.

17. The process as claimed in any one of claims 11 to 16, wherein the lower olefin is at least one member selected from the group consisting of ethylene, propylene, 1-butene, 2-butene and butadiene.

18. The process as claimed in any one of claims 11 to 17, wherein the lower fatty acid is at least one member selected from the group consisting of a formic acid, an acetic acid, a propionic acid, an acrylic acid and a methacrylic acid.
